(19) United States
(12) Patent Application Publication
(10) Pub. No.: US 2002/0077301 A1
(43) Pub. Date: Jun. 20, 2002

(54) Effects of combined administration of farnesyl transferase inhibitors and signal transduction inhibitors

(75) Inventors: George Q. Daley, Weston, MA (US); Russell R. Hoover, Brighton, MA (US)

Correspondence Address: Anne J. Collins, Esq., HAMILTON, BROOK. SMITH & REYNOLDS, P.C., 530 Virginia Road, P.O. Box 9133, Concord, MA, 01742-9133, US

(73) Assignee: Whitehead Institute for Biomedical Research, Cambridge, MA

(21) Appl. No.: 09/971,365

(22) Filed: Oct. 4, 2001

Related U.S. Application Data
(60) Provisional application No. 60/238,240, filed on Oct. 5, 2000.
(60) Provisional application No. 60/238,813, filed on Oct. 6, 2000.

(51) Int. Cl. A61K 39/395; A61K 31/505; A61K 31/7048; A61K 31/277

(52) U.S. Cl. 514/27; 424/155.1; 514/272; 514/520

(57) ABSTRACT

The present invention relates to methods of reducing proliferation of cells, enhancing apoptosis of cells or both in an individual in need thereof, comprising administering to the individual a combination of at least one farnesyl transferase inhibitor (FTI), such as an inhibitor of Ras function, and at least one signal transduction inhibitor (STI) in a therapeutically effective amount, wherein proliferation of cells is reduced and/or apoptosis of cells is enhanced in the individual. In one embodiment, the invention relates to a method of reducing proliferation of STI resistant cells, enhancing apoptosis of STI resistant cells, or both in an individual in need thereof, comprising administering to the individual a combination of at least one FTI and at least one STI in a therapeutically effective amount, wherein proliferation of STI resistant cells is reduced and/or apoptosis of STI resistant cells is enhanced in the individual. The present invention can be used to treat leukemia (e.g., CML) in an individual comprising administering to the individual a combination of at least one FTI and at least one STI in a therapeutically effective amount.

### RELATED APPLICATION(S)

This application claims the benefit of U.S. Provisional Application No. 60/238,240, filed on Oct. 5, 2000 and claims the benefit of U.S. Provisional Application No. 60/238,813, filed on Oct. 6, 2000. The entire teachings of the above applications are incorporated herein by reference.

### BACKGROUND OF THE INVENTION

The BCR/ABL oncoprotein induces chronic myeloid leukemia (CML) by a complex process that involves inappropriate activation of cytokine receptor signaling pathways, altered adhesion properties of hematopoietic progenitors in the bone marrow, and protection against apoptotic cell death. CML is characterized by an initial chronic phase where there is an expansion of differentiated myeloid cells. This relatively indolent phase inevitably progresses to blast crises, which resembles an acute leukemia and is often refractory to standard treatments. The transforming properties of BCR/ABL are dependent on its activated tyrosine kinase, thus considerable effort has been invested towards the identification of kinase directed CML therapies. Among the most specific inhibitors of the ABL tyrosine kinase, STI-571 induces apoptosis in BCR/ABL positive cell lines, inhibits hematopoietic colony formation from CML bone marrow, and eradicates BCR/ABL positive leukemia in mouse models (Druker, B. J., et al., Nat. Med., 2(5):561-566 (1996); le Coutre, P., et al., Blood, 95(5):1758-1766 (2000)). STI-571 is well tolerated and has been effective in clinical trials of chronic phase patients (Druker, B. J., et al., N. Engl. J. Med., 344(14):1031-1037 (2001)). Recently, STI-571 has been approved by the US FDA and is now being marketed as the drug Gleevec™.

Although STI-571 represents a promising therapy for CML, STI-571 intolerance or resistance may confound disease treatment. Indeed, a number of STI-571 resistant BCR/ABL positive cell lines have been described (le Coutre, P., et al., Blood, 95(5):1758-1766 (2000); Mahon, F. X., et al., Blood, 96(3):1070-1079 (2000); Weisberg, E., et al., Blood, 95(11):3498-3505 (2000)) and resistance to STI-571 have been demonstrated in a nude mouse model (Gambacorti-Passerini, C., et al., J. Natl. Cancer inst., 92(20):1641-1650 (2000)). In addition, CML progression is accompanied by secondary genetic alterations (Ahuja, H., et al., J. Clin. Invest., 78(6):2042-2047 (1991); Honda, H., et al., Blood, 95(4):1144-1150 (2000)), thus survival of late stage CML leukemia cells may no longer be dependent on BCR/ABL tyrosine kinase activity. Indeed, STI-571 induced hematological responses have been less dramatic in blast crisis patients compared to what is observed in chronic phase patients (Druker, B. J., et al., N. Engl. J Med., 344(14):1038-1042 (2001); Druker, B. J., et al., N. Engl. J. Med., 344(14):1031-1037 (2001)). Recently, reactivation of BCR/ABL signaling either through mutation or amplification of BCR/ABL has been observed in patients that initially responded to STI-571 but then relapsed (Gorre, M. E., et al., Science, 21:21 (2001); Barthe, C., et al., Science, 293(5538):2163 (2001); Hochhaus, A., et al., Science, 293(5538):2163 (2001)).

Therefore, additional therapies are needed to effectively eradicate cancers which are treated with signal transduction inhibitors that target tyrosine kinases (e.g., receptor, non-receptor), such as BCR/ABL positive leukemia.

### SUMMARY OF THE INVENTION

Described herein are results of assessment of the antiproliferative/pro-apoptotic effects of the combined use/administration of a farnesyl inhibitor (FTI, and a signal transduction inhibitor (STI), on cancer cells. Also described herein are results of assessment of activity of an FTI, such as FTI SCH66336, on STI-resistant cells (e.g., BCR/ABL positive leukemic cells). Results show that the STI-resistant cells are at least as sensitive to FTI SCH66336 as are the parental cells and are likely more sensitive than the parental cells.

Accordingly, the present invention relates to methods of reducing (totally or partially) proliferation of cells, enhancing apoptosis of cells or both, by administering a combination of at least one (one or more) FTI and at least one (one or more) STIs to the cells. FTIs are pharmacologic inhibitors of Ras function. In the methods of the present invention, one or more FTIs can be administered with one or more STIs. In one embodiment, as described herein, an FTI and an STI are administered in combination (e.g., at the same time or sufficiently close in time that they have the desired reducing and/or enhancing effect). In a specific embodiment, the FTI SCH66336 is administered in combination with STI-571. As described herein, results support an enhanced antiproliferative/pro-apoptotic effect in cells which are administered the combination. In particular embodiments of the invention, the FTI is SCH66336 (Schering-Plough) and the STI is STI-571 (Novartis).

In a specific embodiment, the invention relates to a method of reducing proliferation of cells, enhancing apoptosis of cells or both in an individual in need thereof, comprising administering to the individual a combination of at least one FTI and at least one STI in a therapeutically effective amount, wherein proliferation of cells is reduced and/or apoptosis of cells is enhanced in the individual. The combination is administered to an individual, such as a mammal (e.g., a mouse or other rodent; a human in whom cell proliferation is to be reduced and/or apoptosis is to be enhanced, such as in an individual with cancer, such as leukemia (e.g., chronic myelogenous leukemia (CML)).

In another embodiment, the invention relates to a method of reducing proliferation of STI resistant cells, enhancing apoptosis of STI resistant cells, or both in an individual in need thereof, comprising administering to the individual a combination of at least one FTI and at least one STI in a therapeutically effective amount, wherein proliferation of STI resistant cells is reduced and/or apoptosis of STI resistant cells is enhanced in the individual. An example of STI resistant cells are BCR/ABL positive cells.

The present invention also relates to a method of treating leukemia in an individual comprising administering to the individual a combination of at least one FTI and at least one STI in a therapeutically effective amount. In a particular embodiment, the present invention relates to a method of treating chronic myeloid leukemia (CML) in an individual comprising administering to the individual a combination of at least one FTI inhibitor and at least one signal transduction inhibitor in a therapeutically effective amount.

The methods described herein provide additional therapies which can be used to treat cancers which are currently treated with STIs that target tyrosine kinases, such as CML.

### BRIEF DESCRIPTION OF THE DRAWINGS

FIG. 1 is a graphic representation of soft agar colony formation of BaF3-BCR/ABL cells in the presence of SCH66336 and STI-571.

FIG. 2 is a bar graph of activity of FTI SCH66336 on parental and STI-571 resistant BaF3-BCR/ABL cells.

FIG. 3 is a graphic representation of assessment of the effect of FTI on STI activity; results show that FTI potentiates the activity of STI.

FIG. 4 is a bar graph showing selective activity of FTI SCH66336 against hematopoietic colonies from CML patients.

FIG. 5 is a graphic representation of results of assessment of colony formation of BCR/ABL transformed BaF3 cells in soft agar in the presence of STI or STI and 100 nM FTI.

FIGS. 6A and 6B are graphs showing that STI-571 resistance does not correspond to SCH66336 resistance. Parental (FIG. 6A) and STI-571 resistant (FIG. 6B) BaF3-BCR/ABL cells were seeded at 5×10⁴ cells/ml in cytokine free RPMI+10% inactivated FBS in the presence of DMSO (control), FTI SCH66336, or the ABL tyrosine kinase inhibitor STI-571. Viable cells were assessed at daily intervals by dye exclusion.

FIG. 7 is a graph showing that SCH66336 inhibits hematopoietic colony formation from STI-571 resistant patients. Hematopoietic progenitor cells were derived from STI571 naive patients (white bars, N=3) or from patients who are clinically resistant to STI571 (gray bars, N=5) and grown in methylcellulose containing the indicated concentration of SCH66336 or STU571. Numbers are normalized to control (DMSO) and are presented as means +/−S.D. of duplicate plates.

FIGS. 8A-8B are graphs showing that SCH66336 sensitizes BaF3-BCR/ABL cells to STI571 induced apoptosis. Parental BaF3-BCR/ABL cells were pretreated with 1 μM SCH66336 or DMSO (control) for 48 hours then exposed to 1 μM STI571 for an additional 8 hours. Some cells were lysed and subjected to immunoblotting with caspase-3 antibody (FIG. 8A) or analyzed for annexin V staining (FIG. 8B) by FACS using the Apo-Alert kit (Clontech).

FIGS. 9A-9E are graphs showing that SCH66336 and STI571 in combination induce apoptosis in STI571 resistant cells. Parental BaF3-BCR/ABL cells (FIG. 9A), STI571 resistant (R) BaF3-BCR/ABL (FIG. 9B), K562 (FIG. 9C), LAMA-84 (FIG. 9D), and AR230 (FIG. 9E) cells were seeded at 5×10⁴ cells/ml in cytokine free RPMI+10% FBS in the presence of DMSO control or the indicated concentrations of SHC66336, STI571, or a combination of both drugs. Viability of cells was assessed at daily intervals by dye exclusion.

### DETAILED DESCRIPTION OF THE INVENTION

Farnesyltransferase inhibitors (FTIs) have been developed as inhibitors of Ras and thus represent a novel class of molecule directed chemotherapeutic agents. FTIs inhibit the posttranslational addition of a 15-carbon farnesyl group to a C-terminal cysteine residue that is required for Ras to properly localize to the cell membrane (Reuter, C. W., et al., Blood, 96(5):1655-1669 (2000)). Though FTIs effectively block Ras signaling, recent data indicate that Ras may not be the primary target of farnesyltransferase inhibition (Ashar, H. R., et al., J. Biol. Chem., 275(39):30451-30457 (2000); Liu, A., et al., Mol. Cell Biol., 20(16):6105-6113 (2000)). The clinical candidate FTI SCH66336 inhibits the proliferation of several human cancer cell lines and is active against human brain, lung, prostate, pancreas, colon, and bladder tumor xenografts in nude mice (Liu, M., et al., Cancer Res., 58(21):4947-4956 (1998); Feldkamp, M. M., et al., Cancer Res., 61(11):4425-4431 (2001)). A recent Phase I clinical trial showed that SCH66336 inhibits protein farnesylation in vivo and is generally well tolerated (Adjei, A. A., et al., Cancer res., 60(7):1871-1877 (2000)). The anti-leukemic activity of SCH66336 on both cell culture models of BCR/ABL transformation and in mouse models of BCR/ABL positive leukemia has recently been shown (Reichert, A., et al., Blood, 97(5):1399-1403 (2001); Peters, D. G., et al., Blood, 97, in press (2001)). As described herein, SCH66336 inhibits the proliferation of STI571 resistant BCR/ABL positive cell lines and hematopoietic colony formation from CML patients unresponsive to STI-571. As also described herein, SCH66336 potently sensitizes STI-571 resistant cells to STI-571 induced apoptosis. The data described herein indicates that the combined administration of at least one FTI inhibitor and at least one signal transduction inhibitor (STI) to a cell can be used to reduce proliferation and/or enhance apoptosis in the cell. In particular, the combination can be used as a therapy (e.g., STI-571 and SCH66336 combination therapy) to treat cancers, particularly cancers which exhibit STI-571 resistance, such as the later stages of CML.

Described herein is characterization of FTIs, pharmacologic inhibitors of Ras function, including the clinical candidate SCH66336 (Schering-Plough). FTI SCH66336 dramatically sensitizes BCR/ABL transformed cells to apoptosis induced by gamma-irradiation and inhibits cell proliferation and soft agar colony formation. FTI SCH66336 can eradicate BCR/ABL-induced leukemia in mice, making it an attractive candidate for testing against leukemias in human trials. Although STI-571 (Novartis), a targeted inhibitor of the activated tyrosine kinase activity of BCR/ABL, has shown promising results in CML patients in early phase clinical trials, the data described herein indicates that combination therapies will be required to most effectively eradicate cancers such as leukemia.

The present invention provides such combination therapies. In one embodiment, the present invention relates to methods of reducing proliferation of cells, enhancing apoptosis of cells or both in an individual in need thereof, comprising administering to the individual a combination of at least one FTI and at least one STI in a therapeutically effective amount, wherein proliferation of cells is reduced and/or apoptosis of cells is enhanced in the individual.

In another embodiment, the invention relates to a method of reducing proliferation of STI resistant cells, enhancing apoptosis of STI resistant cells, or both in an individual in need thereof, comprising administering to the individual a combination of at least one FTI and at least one STI in a therapeutically effective amount, wherein proliferation of STI resistant cells is reduced and/or apoptosis of STI resistant cells is enhanced in the individual. An example of STI resistant cells are BCR/ABL positive cells.

The present invention provides additional methods of treatment. The combined administration of at least one FTI and at least one STI can be used to treat a disease which requires inhibition of proliferation and/or enhanced apoptosis in cells. For example, the combined administration of at least one FTI and at least one STI can be used to treat cancers (solid cancers (e.g., tumor), non-solid cancers (e.g., leukemia)), particularly cancers whose growth is dependent on activated tyrosine kinase (e.g., receptor, non-receptor) activity. Examples of such cancers include leukemia (e.g., BCR/ABL positive leukemia, CML), lung cancer, glioma, breast cancer (e.g., her2 and/or neu positive breast cancer), epithelial cancer, c-kit positive cancer (e.g., gastrointestinal stromal tumor (GIST)), platelet derived growth factor (PDGF) associated with glioforma and dermatofibrosarcoma protuberans.

In a particular embodiment, the present invention can be used to treat leukemia in an individual comprising administering to the individual a combination of at least one FTI and at least one STI in a therapeutically effective amount. In another embodiment, the present invention can be used to treat chronic myeloid leukemia (CML) in an individual comprising administering to the individual a combination of at least one FTI and at least one STI in a therapeutically effective amount.

A variety of FTIs can be used in the methods of the present invention (e.g., see Reuter, C. W., et al., Blood, 96(5):1655-1669 (2000)). In one embodiment, the FTI is an inhibitor of Ras function, such as SCH66336 (Schering-Plough); Reuter, C. W., et al., Blood, 96(5):1655-1669 (2000); Liu, M., et al., Cancer Res., 58(21):4947-4956 (1998)). Other FTIs which can be used in the methods of the present invention include, for example, SCH44342 (Reuter, C. W., et al., Blood, 96(5):1655-1669 (2000)); R115777 (End, D. W., et al., Cancer Res., 61(1):131-137 (2001); Reuter, C. W., et al., Blood, 96(5):1655-1669 (2000)), L778,123 (Buser, C. A., et al., Anal. Biochem., 290(1):126137 (2001); Reuter, C. W., et al., Blood, 96(5):1655-1669 (2000)) and Manumycin (Hara, M., et al., Proc. Natl. Acad. Sci., USA, 90(6):2281-2285 (1993)).

Similarly, there are a variety of STIs which can be used in the methods of the present invention. In one embodiment, STIs which inhibit tyrosine kinase (receptor tyrosine kinase, non-receptor tyrosine kinase) are used in the methods of the present invention (e.g., see Al-Obeidi, F. A., et al., Oncogene, 19(49):5690-5701 (2000); Levitzki and Gazit, Science, 267:1782-1788 (1995)). STIs which inhibit a tyrosine kinase include, for example, STIs that inhibit a tyrosine kinase of the platelet derived growth factor (PDGF) receptor family, the EGF receptor kinase family, the ABL kinase family, the vegf kinase family and the src kinase family. In one embodiment, the STI is STI-571 (Novartis). Other STIs which can be used in the methods of the present invention include, for example, ZD-1839 (C225, an inhibitor of EGFR) (Goldstein, N. I., et al., Clin, Cancer Res., 1(11):1311-1318 (1995); Al-Obeidi, F. A., et al., Oncogene, 19(49):5690-5701 (2000)), SU5416 (Fong, T. A., et al., Cancer Res., 59(1):99-106 (1999); Al-Obeidi, F. A., et al., Oncogene, 19(49):5690-5701 (2000)), Genistein (Al-Obeidi, F. A., et al., Oncogene, 19(49):5690-5701 (2000)), Trastuzumab (herceptin) (Baselga, J., et al., Cancer Res., 58(13):2825-2831 (1998); Al-Obeidi, F. A., et al., Oncogene, 19(49):5690-5701 (2000)), Benzylidene malononitrile (tyrphostins and their analogues) (Al-Obeidi, F. A., et al., Oncogene, 19(49):5690-5701 (2000)), and Phenylamino-pyrimidines (Al-Obeidi, F. A., et al., Oncogene, 19(49):5690-5701 (2000)).

An FTI and an STI are administered in combination. Therefore, the FTI(s) and the STI(s) can be administered at the same time (simultaneously) or sufficiently close in time (subsequently to one another, e.g., prior, preferably just prior, or after, preferably just after, one another) so that they have the desired effect (e.g., reduced proliferation of cells, enhanced apoptosis of cells or both).

The FTI(s) and STI(s) either alone or in a combined formulation are administered in a therapeutically effective amount which is an amount sufficient to have the desired effect(s), such as an amount sufficient to reduce proliferation of cells, enhance apoptosis of cells or both in an individual in need thereof. Administration of a therapeutically effective amount generally results in improved condition of the individual over time. The amount of the FTI(s) and the STI(s) used in the methods of the present invention will vary depending on a variety of factors including the size, age, body weight, general health, sex, and diet of the individual, the time of administration, and the duration or particular qualitites of the disease state, and can be determined by a skilled practitioner. The FTI(s) and the STI(s) can be administered in a single dose or in multiple doses and the order of administration can vary. Furthermore, it is not necessary that the FTI(s) and STI(s) be administered via the same route.

The combination of FTI(s)and STI(s) is administered to an individual, such as a mammal in whom cell proliferation is to be reduced and/or apoptosis is to be enhanced, such as in an individual with cancer (e.g., leukemia, CML). For example, the FTI(s) and STI(s) can be administered to a murine (e.g., a mouse, rat or other rodent), a primate (e.g., a human or monkey), a canine, a feline, a bovine or a porcine individual.

Administration of the FTI(s) and STI(s) can be achieved by a variety of routes, such as by parenteral routes (e.g., intravenous, intraarterial, intramuscular subcutaneous injection), topical, inhalation (e.g., intrabronchial, intranasal or oral inhalation or intranasal drops), oral (e.g., dietary), rectal or other route.

Formulation will vary according to the route of administration selected (e.g., solution, emulsion). An appropriate composition comprising the FTI(s), the STI(s) or a combination of both to be administered can be prepared in a physiologically acceptable carrier. For solutions or emulsions, suitable carriers include, for example, aqueous or alcoholic solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See, for example, Remington's Pharmaceutical Sciences, 17^{th} edition, Mack Publishing Co., PA, 1985). For inhalation, the inhibitor(s) of Ras, STI(s) or combination of both can be solubilized and loaded into a suitable dispenser for administration (e.g., an atomizer, nebulizer or pressurized aerosol dispenser).

### Exemplification

### Example 1

### Effect of SCH66336 Alone and in Combination with STI-571 on Colony Formation

### Colony Formation in Soft Agar

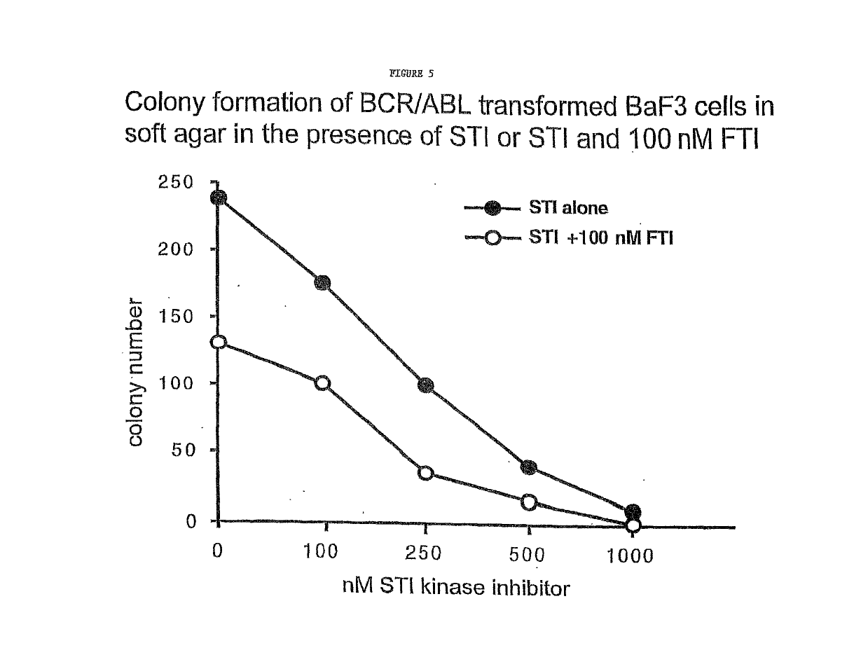

To determine the effect of SCH66336 alone and in combination with STI-571 on the ability of BCR/ABL-BaF3 cells to form macroscopic colonies in soft agar, 10,000 cells were plated in each 3.5 cm well of a 6-well dish containing RPMI+10% inactivated fetal bovine serum (FBS) supplemented with 0.3% bacto-agar. SCH66336 (Shering-Plough) and/or STI-571 (Novartis) were added to the media from a 10 mM (millimolar) dimethyl sulfoxide (DMSO) stock to reach final concentrations specified. Macroscopic colonies were counted in duplicate plates on day 10. In some cases colony numbers were normalized by dividing the number of colonies under a given condition by the number of colonies formed in the presence of no drug (DMSO alone). See FIGS. 1, 2, 3 and 5. Methylcellulose colony assays of human primary cells.

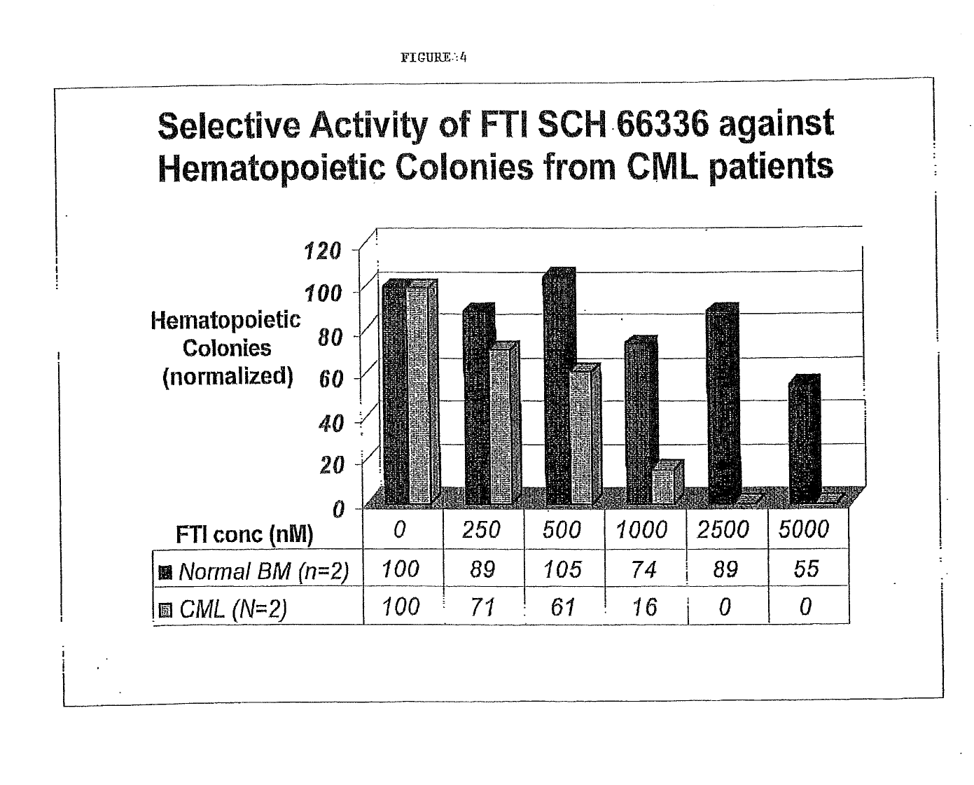

Total bone marrow cells from human normals and CD34+ bone marrow and peripheral blood cells from human CML patients were plated in methylcellulose containing human growth factors (IL-3, IL-6, stem cell factor, erythropoietin; MethoCult GF h4434, Stem Cell Technologies). SCH66336 (Shering-Plough) and/or STI-571 (Novartis) were added to the media from a 10 mM (millimolar) dimethyl sulfoxide (DMSO) stock to reach final concentrations specified. Initial experiments were done to determine the optimal seeding density such that plates contained between 100-150 colonies/35 mm petri dish (2-5×10⁵ cells/plate for whole bone marrow and 2×10³ cells/plate for CD34+ cells)in the absence of drug (DMSO control). Blinded colony counts were performed on duplicate plates on day 14 using an inverted microscope. See FIG. 4.

### Results

The antiproliferative/pro-apoptotic effects of FTI SCH66336 in combination with ST-571 have been assessed on a murine in vitro model of BCR/ABL transformation, BaF3-BCR/ABL. Results of a representative experiment are shown in FIG. 1. In the experiment, BaF3-BCR/ABL cells are grown in soft agar in the presence of increasing concentrations of either STI-571, FTI SCH66336, or both. Results support the conclusion that inhibition of soft agar colony number by STI-571 is enhanced by the presence of FTI SCH66336. The IC50 of STI-571 and FTI SCH 66336 is approximately 200 nM and 100 nM, respectively. However, a combination of STI571 and SCH66336 at their respective IC₅₀ doses results in an inhibition of soft agar colony formation to about 21% of control, suggesting that the two agents have at least an additive effect.

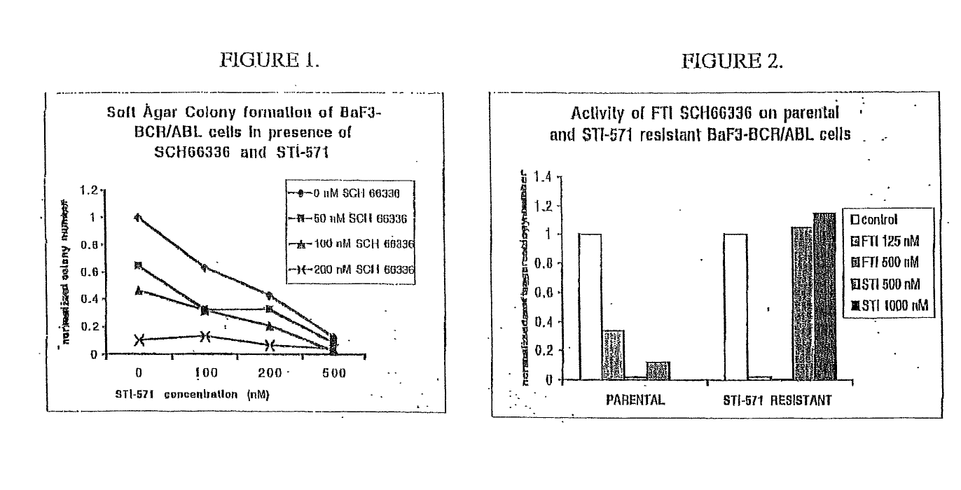

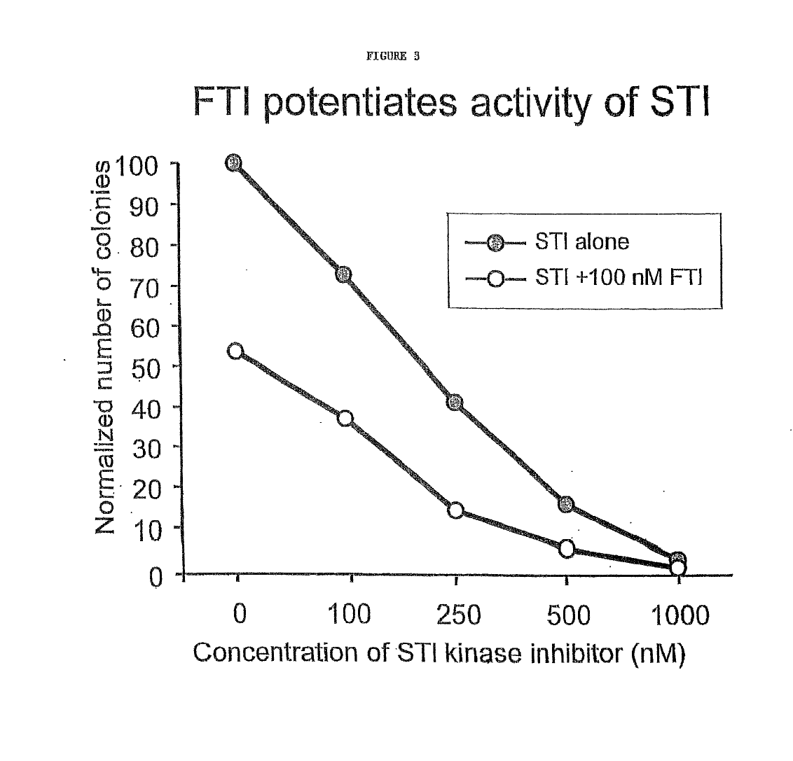

Resistance to STI-571 has been demonstrated in a number of mouse and human BCR/ABL positive cell lines including, BaF3-BCR/ABL, K562, AR230, and LAMA84. FIG. 2 shows the effects of FTI SCH66336 on an STI-571 resistant clone of a BaF3-BCR/ABL. STI-571 does not decrease soft agar colony formation in the resistant clone, even at concentrations that eliminate colony formation of the parental cells. By contrast, the STI-571 resistant cells are as sensitive, if not more so, to FTI SCH66336 as the parental BaF3-BCR/ABL cell line. This experiment has also been repeated with STI-571 resistant and parental K562 and AR230 cells with similar results.

FIG. 3 shows results that demonstrate that FTI potentiates the activity of STI on colony formation. FIG. 4 shows results of assessment of the activity of FTI SCH66336 against hematopoietic colonies from CR/ABL transformed BaF3 cells in soft agar in the presence of STI alone or STI and 100 nM FTI. Colony formation is inhibited to a greater extent in the presence of the combination of compounds than in the presence of STI only.

### Example 2

### Overcoming STI-571 Resistance with the Farnesyltransferase Inhibitor SCH66336

### Methods and Materials

### Cell lines

Derivation of STI-571 resistant (R) BaF3-BCR/ABL, AR230, LAMA84, and K562 cell lines has been described previously (Mahon, F. X., et al., Blood, 96(3):1070-1079 (2000); Weisberg, E., et al., Blood, 95(11):3498-3505 (2000)). Both parental and STI-571 resistant cell lines were maintained in RPMI 1640 supplemented with 10% inactivated fetal bovine serum. For the STI-571 resistant cell lines the media was also supplemented with 500 nM STI-571.

### Compounds and Reagents

The farnesyltransferase inhibitor (FTI) SCH66336 was a gift of Schering-Plough Research Institute (Kenilworth, N.J.) and the abl specific kinase inhibitor STI-571 was a gist of Novartis (Basel, Switzerland). Both compounds were stored as 10 mM stocks in DMSO. Incubation times used for SCH66336 are longer than those for STI-571 SCG6636 acts posttranslationally, thus cellular effects are not generally seen until 48 hours after drug addition. Monoclonal antibody against caspase-3 (CPP32) was purchased from Santa Cruz Biotechnology (Santa Cruz, Calif.).

### Measurement of Apoptosis and Cell Viability

Apoptosis was measured in cells after incubation with drugs by staining for annexin-positive cells using the ApoAlert Annexin V kit (Clontech, Palo Alto, Calif.). Flow cytometry analysis was performed using Cell Quest (Becton Dickinson, Franklin Lakes, N.J.). Cell viability was measured at daily intervals using trypan blue dye exclusion.

### Immunoblotting

Cells were washed in cold PBS and lysed in NP-40 lysis buffer (1% NP-40, 150 mM NaCl, 20 mM Tris (pH 7.4), 10% glycerol, 10 mM NaVO3, 1 mM ZnCl₂, 1 mM MgCl₂, and 2 mM PMSF). Approximately 50 ug of protein were separated by SDS-PAGE and electrophoretically transferred to nitrocellulose membrane using standard protocols. Membranes were incubated with primary and secondary antibodies in TBST buffer (20 mM Tris-HCl [pH 7.4], 500 mM NaCl, 0.01% Tween 20) containing 5% dry milk. Immunoreactivity was detected by enhanced chemiluminescence.

### Patient Material and Methylcellulose Colony Assays

Low density mononucleocytes were isolated from either fresh or cryo-preserved peripheral blood using Lymphoprep (Nycomed, Oslo, Norway). Cells from STI-571 resistant patients were plated in Iscoves' methylcellulose medium (Methocult H4330; Stemcell Technologies Inc., Vancouver, Canada) supplemented with 20 ng/ml recombinant hIL-3, hG-CSF, hGM-CSF, hIL-6 (Amgen, Thousand Oaks, Calif.) and 100 ng/ml Flt3 ligand R&D Systems Abingdon, Oxon UK). Cells from STI-571 naive patients were plated Methocult H4434 (Stemcell Technologies Inc., Vancouver, Canada), which contains hSCF in place of Flt3 ligand. All clonogenic assays were performed in duplicate.

### Results

### SCG66336 inhibits the proliferation of STI-571 cell lines

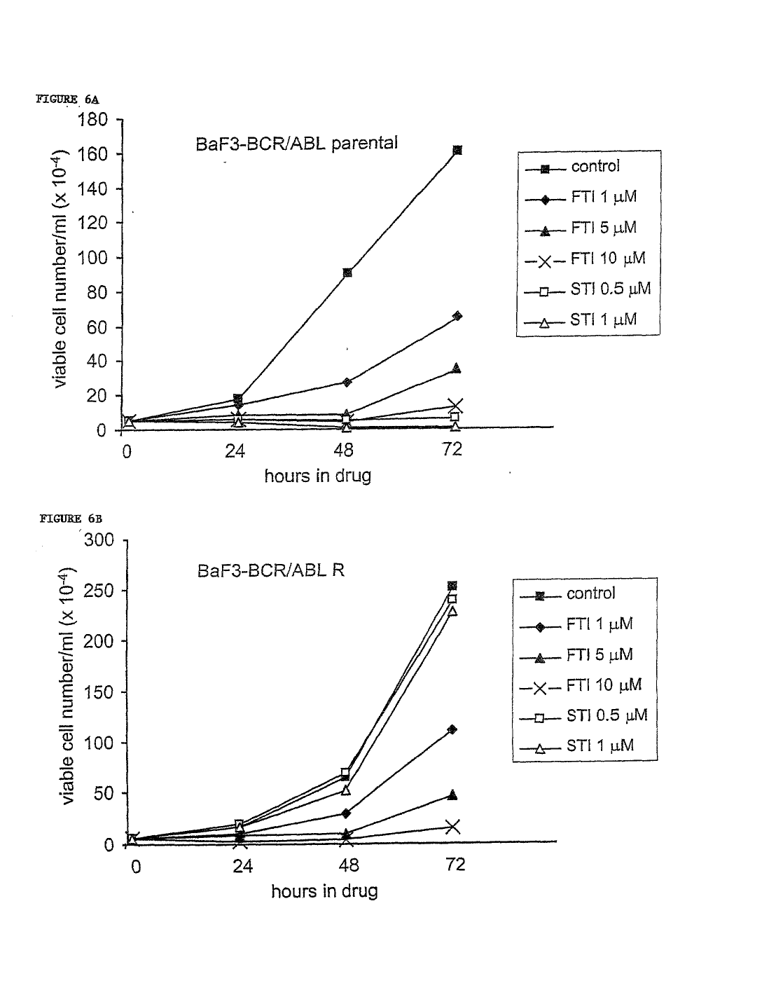

The antiproliferative effect of the FTI SCH66336 on BCR/ABL positive leukemic cell lines has recently been shown (Peters, D. G., et al., Blood, 97, in press (2001)). To determine whether resistance to the tyrosine kinase inhibitor STI-571 correlates with resistance to SCH66336, parental and STI-571 resistant cell lines were placed in liquid culture containing either DMSO (control), 0.5 or 1.0 μM STI-571, or increasing concentrations of SCH66336. FIGS. 6A-6B shows that while STI-571 has no effect on the growth of STI-571 resistant BaF3-BCR/ABL cells, SCH66336 induces a dose-dependent inhibition of proliferation. The anti-proliferative effects of SCH66336 on BaF3-BCR/ABL cells are a consequence of G2/M blockade (Peters, D. G., et al., Blood, 97, in press (2001)). No difference in the effects of SHC66336 between the growth of parental and STI-571 resistant BaF3-BCR/ABL cells could be shown (FIGS. 6A-6B). Likewise, SCH66336 inhibits the proliferation of STI-571 resistant K562, AR230, and LAMA84 cells in a similar manner as the respective parental (STI-571 sensitive) cell lines. STI-571 resistance in a BaF3-BCR/ABL, LAMA84, and AR230 is due to amplification of the BCR/ABL gene, a phenomenon that corresponds to STI-571 resistance in patients (Gorre, M. E., et al., Science, 21:21 (2001)). Thus SCH66336 is effective on BCRIABL positive leukemic cells despite increased levels of BCR/ABL sufficient for STI-571 resistance, indicating that resistance does not correspond to resistance to SCH66336.

### SCH66336 Inhibits Colony Formation of Hematopoietic Progenitors from STI-571 Unresponsive Patients

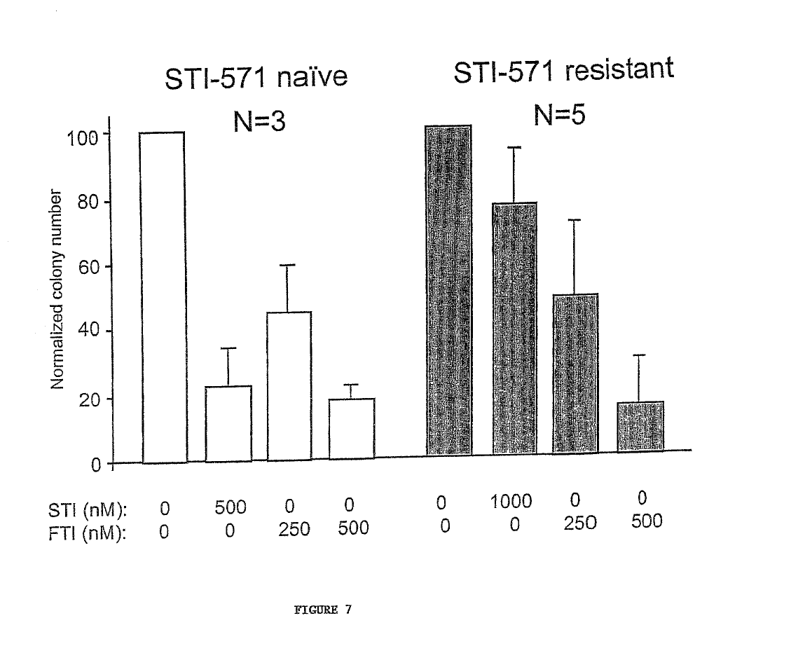

To determine whether SCH66336 was effective against BCR/ABL positive leukemic cells from patients unresponsive to STI-571, primary cells from 5 CML patients that had relapsed while on STI-571 therapy were cultured in methylcellulose in presence of increasing concentrations of SCH66336. Cellular resistance to STI-571 was indicated by robust hematopoietic colony formation in the presence of 1 μM STI-571, more than double the IC₅₀ for hematopoietic cells from CML patients naive to STI-571 treatment (FIG. 7). Colony formation of STI-571 resistant hematopoietic progenitors was significantly inhibited by SHC66336, with an IC₅₀ of between 250 and 500 nM, a value that is similar for hematopoietic cells from STI-571 naive CML patients (FIG. 7, and Peters, D. G., et al., Blood, 97, in press (2001)). These results indicate that the growth of human CML cells from patients unresponsive to STI-571 is inhibited by SCH66336 treatment.

### SCH66336 Sensitizes BCR/ABL Positive Cells to STI-571 Induced Apoptosis

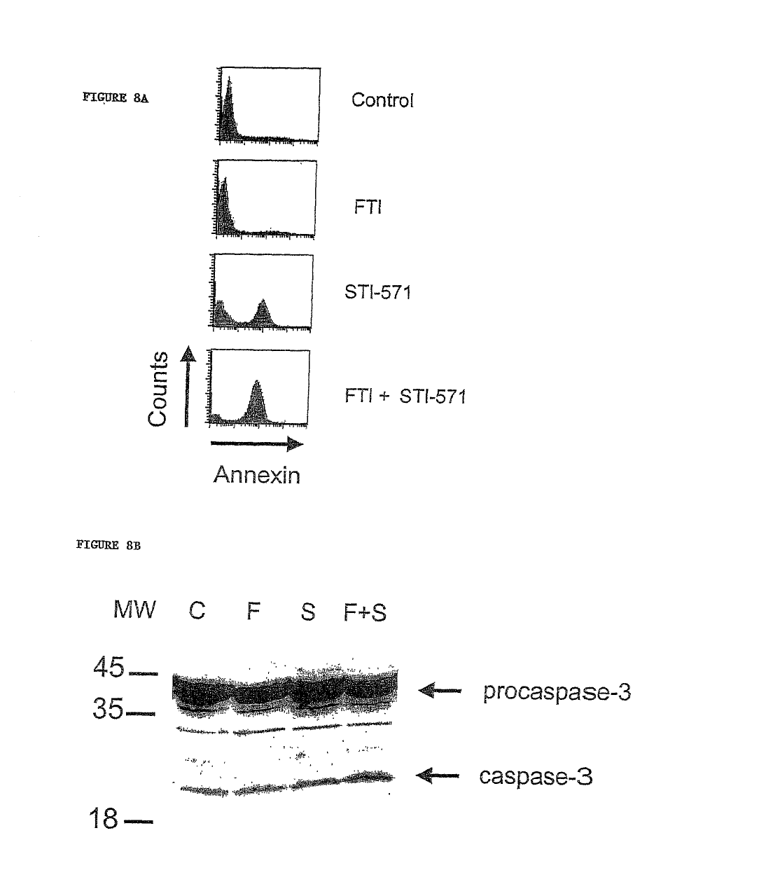

SCH66336, although not an inducer of apoptosis, sensitizes BCR/ABL positive cells to apoptotic signals such as γ-irradiation and serum starvation (Peters, D. G., et al., Blood, 97, in press (2001)). To determine whether SCH66336 would sensitize BCR/ABL positive cells to STI-571 induced apoptosis, parental BaF3-BCR/ABL cells were incubated in the presence of either DMSO (control), 2.0 uM SCH66336, 1 uM STI-571, or a combination of drugs and levels of annexin V staining were determined using the ApoAlert Annexin V kit. Low levels of annexin V staining were found on BaF3-BCR/ABL cells exposed to DMSO or SCH66336 (48 hours), while approximately 50% of the BaF3-BCR/ABL cells are annexin V positive after 12 hours in the presence of STI-571 (FIG. 8A). SCH66336 potentiates STI-571 induced apoptosis as shown in the bottom panel of FIG. 8A, where 80% of BaF3-BCR/ABL cells are annexin V positive in the presence of both drugs. This synergistic effect on apoptosis is also demonstrated by analyzing the cleavage of inactive procaspase-3 into caspase-3. Immunoblotting of protein lysates from cells treated with either DMSO or SCH66336 (FIG. 8A, lanes 1 and 2, respectively) show low levels of active caspase-3. Treatment with STI-571 increases levels of active caspase-3, which is further increased in the presence of both drugs (FIG. 8B, lanes 3 and 4). SCH66336 and STI-571 combination inhibits cell viability of STI-571 resistant cells with BCR/ABL amplification

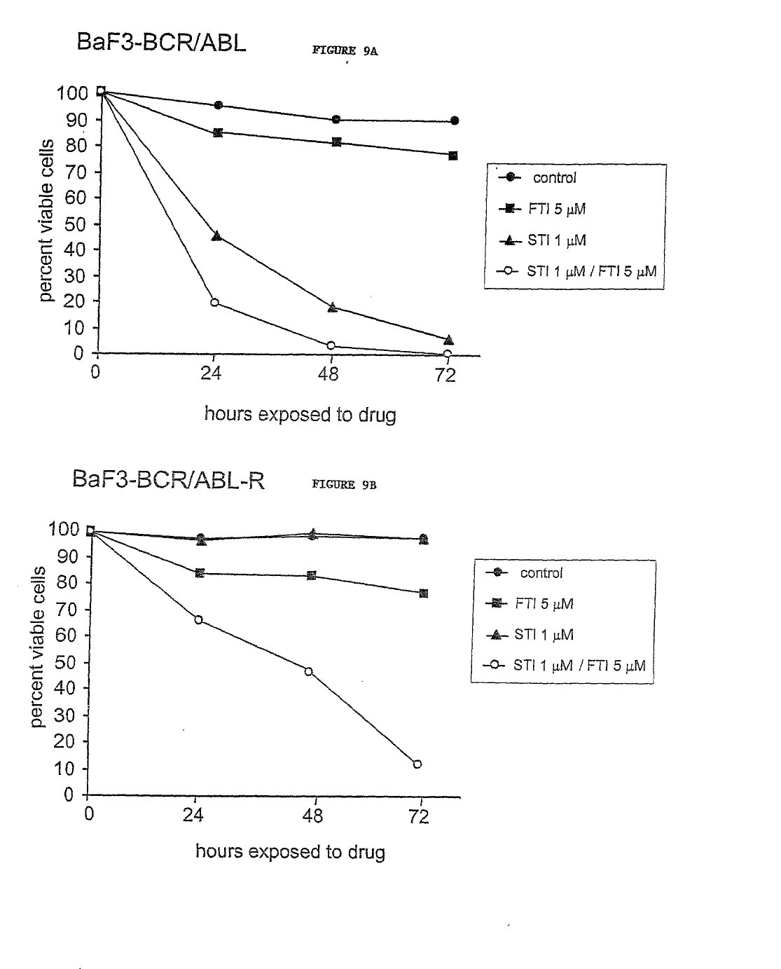

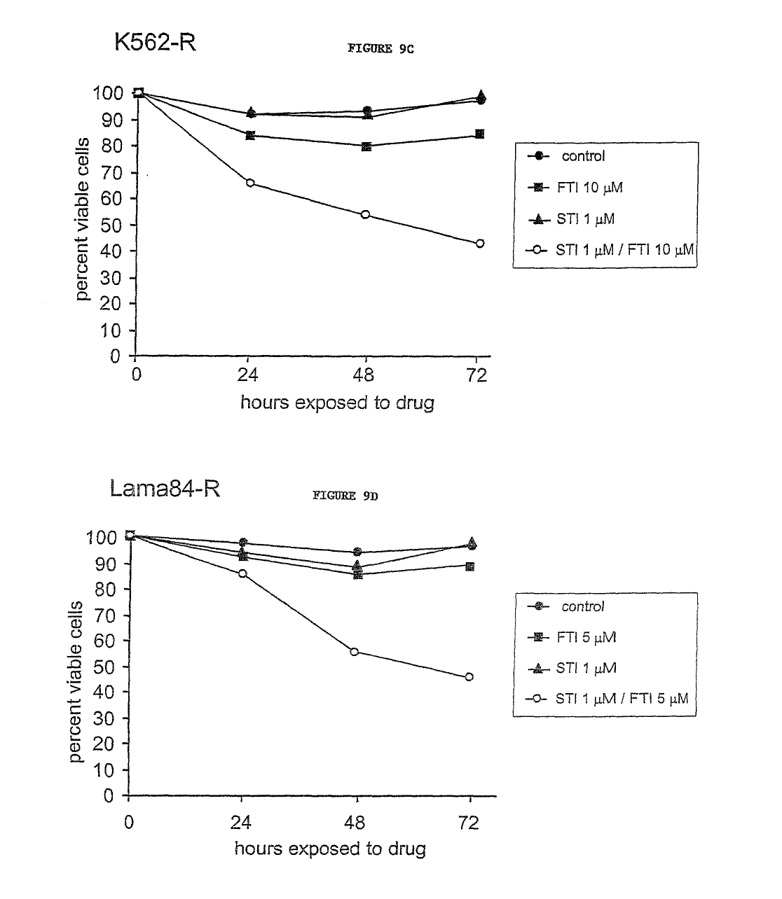

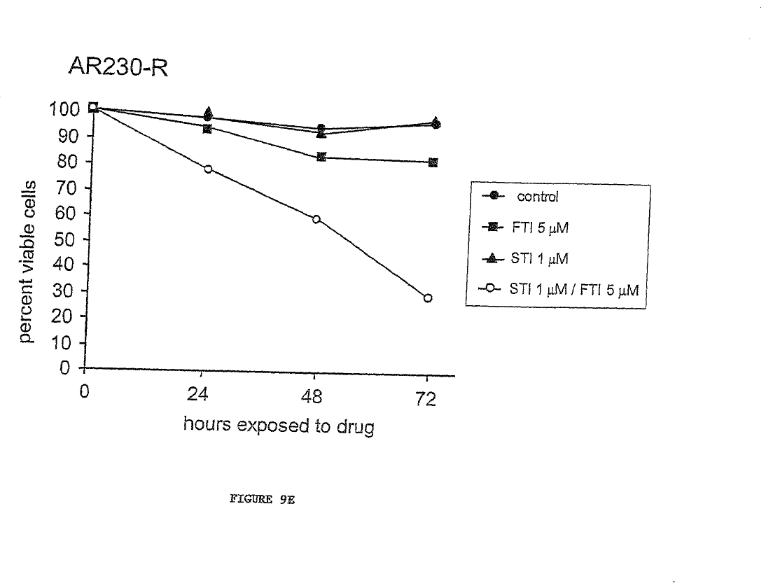

Although SCH66336 inhibits cell proliferation, it has little effect on cell viability at concentrations up to 5 μM on either STI-571 sensitive or resistant cell lines (FIG. 9A-9E). However, when SCH66336 and STI-571 are combined, STI-571 resistant cells undergo a dramatic decrease in cell viability (FIG. 9B-9E). STI-571 resistant K562 erythroleukemia cells require higher concentration of SCH66336 as these cells express high levels of the MDR gene. While cells treated with SCH66336 alone remain mostly viable and proliferate, although at a reduced rate, after approximately 96-128 hours of combined SCH66336 and STI-571 drug treatment STI-571 resistant cells are mostly non-viable and do not recover.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of reducing proliferation of cells, enhancing apoptosis of cells or both in an individual in need thereof, comprising administering to the individual a combination of at least one farnesyl transferase inhibitor (FTI) and at least one signal transduction inhibitor (STD in a therapeutically effective amount, wherein proliferation of cells is reduced and/or apoptosis of cells is enhanced in the individual.

2. The method of claim 1, wherein the individual has cancer.

3. The method of claim 2, wherein the cancer is leukemia.

4. The method of claim 3, wherein the leukemia is chronic myeloid leukemia (CML).

5. The method of claim 1 wherein the cells are BCW/ABL positive cells.

6. The method of claim 1 wherein the farnesyl transferase inhibitor is an inhibitor of Ras function.

7. The method of claim 1 wherein the farnesyl transferase inhibitor is selected from the group consisting of: SCH66336; SCH44342; R115777; L778,123; and Manumycin.

8. The method of claim 1 wherein the signal transduction inhibitor inhibits a tyrosine kinase selected from the group consisting of: a tyrosine kinase of the platelet derived growth factor (PDGF) receptor family, the EGF receptor kinase family, the ABL kinase family, the vegf kinase family and the src kinase family.

9. The method of claim 1 wherein the signal transduction inhibitor is selected from the group consisting of: STI-571; ZD-1839; SU5416; Genistein; Trastuzumab; Benzylidene malononitrile; and Phenylamino-pyrimidines.

10. The method of claim 1 wherein the FTI is SCH66336 and the STI is STI-571.

11. A method of reducing proliferation of STI resistant cells, enhancing apoptosis of STI resistant cells, or both in an individual in need thereof, comprising administering to the individual a combination of at least one FI and at least one STI in a therapeutically effective amount, wherein proliferation of STI resistant cells is reduced and/or apoptosis of STI resistant cells is enhanced in the individual.

12. The method of claim 11 wherein the STI resistant cells are BCR/ABL positive cells.

13. The method of claim 11, wherein the individual has cancer.

14. The method of claim 13, wherein the cancer is leukemia.

15. The method of claim 14, wherein the leukemia is chronic myeloid leukemia (CML).

16. The method of claim 11 wherein the FTI is an inhibitor of Ras function.

17. The method of claim 11 wherein the FTI is selected from the group consisting of: SCH66336; SCH44342; R115777; L778,123; and Manumycin.

18. The method of claim 11 wherein the signal transduction inhibitor inhibits a tyrosine kinase selected from the group consisting of: a tyrosine kinase of the platelet derived growth factor (PDGF) receptor family, the EGF receptor kinase family, the ABL kinase family, the vegf kinase family and the src kinase family.

19. The method of claim 11 wherein the signal transduction inhibitor is selected from the group consisting of: STI-571; ZD-1839; SU5416; Genistein; Trastuzumab; Benzylidene malononitrile; and Phenylamino-pyrimidines.

20. The method of claim 11 wherein the FTI is SCH66336 and the STI is STI-571.

21. A method of treating leukemia in an individual comprising administering to the individual a combination of at least one FTI and at least one STI in a therapeutically effective amount.

22. The method of claim 21, wherein the leukemia is chronic myeloid leukemia (CML).

23. The method of claim 21 wherein the FTI is an inhibitor of Ras function.

24. The method of claim 21 wherein the FTI is selected from the group consisting of: SCH66336; SCH44342; R115777; L778,123; and Manumycin.

25. The method of claim 21 wherein the signal transduction inhibitor inhibits a tyrosine kinase selected from the group consisting of: a tyrosine kinase of the platelet derived growth factor (PDGF) receptor family, the EGF receptor kinase family, the ABL kinase family, the vegf kinase family and the src kinase family.

26. The method of claim 21 wherein the signal transduction inhibitor is selected from the group consisting of: STI-571; ZD-1839; SU5416; Genistein; Trastuzumab; Benzylidene malononitrile; and Phenylamino-pyrimidines.

27. The method of claim 21 wherein the FTI is SCH66336 and the STI is STI-571.

28. A method of treating chronic myeloid leukemia (CML) in an individual comprising administering to the individual a combination of at least one FTI and at least one STI in a therapeutically effective amount.

29. The method of claim 28 wherein the FTI is an inhibitor of Ras function.

30. The method of claim 28 wherein the FTI is selected from the group consisting of: SCH66336; SCH44342; R115777; L778,123; and Manumycin.

31. The method of claim 28 wherein the signal transduction inhibitor inhibits a tyrosine kinase selected from the group consisting of: a tyrosine kinase of the platelet derived growth factor (PDGF) receptor family, the EGF receptor kinase family, the ABL kinase family, the vegf kinase family and the src kinase family.

32. The method of claim 28 wherein the signal transduction inhibitor is selected from the group consisting of: STI-571; ZD-1839; SU5416; Genistein; Trastuzumab; Benzylidene malononitrile; and Phenylamino-pyrimidines.

33. The method of claim 28 wherein the FTI is SCH66336 and the STI is STI-571.
